(19) United States
(12) Patent Application Publication
(10) Pub. No.: US 2021/0398693 A1
(43) Pub. Date: Dec. 23, 2021

(54) METHOD FOR DRUG RECOMMENDATION, ELECTRONIC DEVICE AND COMPUTER-READABLE STORAGE MEDIUM

(71) Applicant: BOE Technology Co.,Ltd., Beijing (CN)

(72) Inventor: Yafei Dai, Beijing (CN)

(21) Appl. No.: 17/255,002

(22) PCT Filed: Jun. 17, 2020
(86) PCT No.: PCT/CN2020/096583

§ 371 Date: Dec. 22, 2020

(30) Foreign Application Priority Data
Jun. 17, 2019 (CN) 201910521202.3

(51) Int. Cl. G16H 70/40; G06F 16/9535; G06F 16/33; G06N 3/02

(52) U.S. Cl. CPC G16H 70/40; G06F 16/9535; G06Q 30/0631; G06N 3/02; G06F 16/334

(57) ABSTRACT

The present disclosure proposes a method for drug recommendation, an electronic device and a computer-readable storage medium, wherein the method comprises: receiving information of a specified disease and a name of a first drug for the specified disease input by a user; obtaining information of the first drug associated with the name of the first drug and information of a plurality of second drugs associated with the information of the specified disease from a pre-established drug database; determining a text semantic similarity between the information of the first drug and information of each of the plurality of second drugs; determining information of a recommended drug from the information of the plurality of second drugs based on the text semantic similarity; and outputting the information of the recommended drug.

### CROSS-REFERENCE TO RELATED APPLICATION

This application is a Section 371 National Stage Application of International Application No. PCT/CN2020/096583, filed on Jun. 17, 2020, entitled “METHOD FOR DRUG RECOMMENDATION, ELECTRONIC DEVICE AND COMPUTER-READABLE STORAGE MEDIUM”, which claims priority to the Chinese Patent Application No. 201910521202.3, filed on Jun. 17, 2019, which are incorporated herein by reference in their entireties.

### TECHNICAL FIELD

The present disclosure relates to the field of drug recommendation, and more particularly, to a method for drug recommendation, an electronic device and a computer-readable storage medium.

### BACKGROUND

Generally, users may choose to purchase several commonly used drugs in pharmacies. For example, when catching a cold, users usually purchase Contac NT, Gankang, etc. This may lead to drug resistance, and the development of drug resistance makes effects of normal doses of drugs no longer as desired, or even makes the drug completely ineffective, being unbeneficial to the recovery of the patient.

### SUMMARY

The present disclosure provides a method for drug recommendation, comprising:

receiving information of a specified disease and a name of a first drug for the specified disease input by a user;

obtaining information of the first drug associated with the name of the first drug and information of a plurality of second drugs associated with the information of the specified disease from a pre-established drug database;

determining a text semantic similarity between the information of the first drug and information of each of the plurality of second drugs;

determining information of a recommended drug from the information of the plurality of second drugs based on the text semantic similarity; and outputting the information of the recommended drug.

For example, before receiving the information of the specified disease and the name of the first drug for the specified disease input by the user, further comprising:

obtaining information of a plurality of diseases and drug data for each of the plurality of diseases from a drug information source; and

for each of the plurality of diseases, extracting information of a drug for the disease from the drug data, and storing information of the disease in association with the information of the drug for the disease to establish the drug database.

For example, the information of the drug includes at least one of a name of the drug, efficacy information of the drug and chemical composition information of the drug.

For example, the information of the drug includes the name of the drug, the efficacy information of the drug and the chemical composition information of the drug, and storing the information of the disease in association with the information of the drug for the disease includes:

setting a disease identification of the disease for the information of the disease and setting a drug identification of each drug for the disease for a name of said each drug for the disease;

establishing a mapping table between the disease identification and the drug identification for each drug; and

storing the drug identification for each drug in the mapping table and the efficacy information and the chemical composition information of the drug in form of a dictionary.

For example, obtaining the information of the first drug associated with the name of the first drug and the information of the plurality of second drugs associated with the information of the specified disease from the pre-established drug database includes:

in the pre-established drug database, searching for a drug identification for the first drug based on the name of the first drug, and obtaining efficacy information and chemical composition information of the first drug based on the drug identification for the first drug; and

searching for a disease identification for the specified disease based on the information of the specified disease, determining drug identifications for a plurality of second drugs associated with the disease identification for the specified disease, and obtaining a name, efficacy information and chemical composition information of each of the plurality of second drugs based on a drug identification for said each of the plurality of second drugs.

For example, determining the text semantic similarity between the information of the first drug and the information of each of the plurality of second drugs includes:

calculating an efficacy similarity between the efficacy information of the first drug and the efficacy information of said each second drug;

calculating a composition similarity between the chemical composition information of the first drug and the chemical composition information of said each second drug; and

calculating the text semantic similarity between the information of the first drug and the information of said each second drug according to the efficacy similarity and the composition similarity.

For example, the efficacy information of the first drug includes text describing efficacy of the first drug, the efficacy information of the second drug includes text describing efficacy of the second drug, and calculating the efficacy similarity between the efficacy information of the first drug and the efficacy information of said each second drug includes:

performing word segmentation and keyword recognition processing on the text describing the efficacy of the first drug to generate a first word vector sequence of a first keyword for the efficacy information of the first drug;

performing word segmentation and keyword recognition processing on the text describing efficacy of each second drug to generate a second word vector sequence of a second keyword for the efficacy information of each second drug; and

inputting the first word vector sequence and the second word vector sequence into a first neural network model, to calculate the efficacy similarity.

For example, the chemical composition information of the first drug includes text describing chemical composition of the first drug, the chemical composition information of the second drug includes text describing chemical composition of the second drug, and calculating the composition similarity between the chemical composition information of the first drug and the chemical composition information of said each second drug includes:

performing word segmentation and keyword recognition processing on the text describing the chemical composition of the first drug to generate a third word vector sequence of a third keyword for the chemical composition information of the first drug;

performing word segmentation and keyword recognition processing on the text describing the chemical composition of said each second drug to generate a fourth word vector sequence of a fourth keyword for the chemical composition information of said each second drug; and

inputting the third word vector sequence and the fourth word vector sequence into a second neural network model, to calculate the composition similarity.

For example, calculating the text semantic similarity between the information of the first drug and the information of said each second drug according to the efficacy similarity and the composition similarity includes:

calculating an average value of the efficacy similarity and the composition similarity between the first drug and said each second drug as the text semantic similarity between the information of the first drug and the information of said each second drug.

For example, determining the information of the recommended drug from the information of the plurality of second drugs based on the text semantic similarity includes:

obtaining a highest similarity of the text semantic similarities to the information of the first drug as a target text semantic similarity; and

from the information of the plurality of second drugs, determining information of a second drug having the target text semantic similarity to the information of the first drug as the information of the recommended drug.

For example, the information of the specified disease includes a name of the specified disease.

The embodiment of the present disclosure also provide an electronic device, comprising:

a processor; and

a memory, configured to store executable instructions for the processor;

wherein, the processor is configured to execute the method for drug recommendation described above.

The embodiment of the present disclosure also provide a computer-readable storage medium, storing instructions thereon, wherein the instructions, when executed by a processor, cause the processor to execute the method for drug recommendation described above.

### BRIEF DESCRIPTION OF THE ACCOMPANYING DRAWINGS

FIG. 1 shows a flowchart of steps of a method for drug recommendation according to an embodiment of the present disclosure;

FIG. 2A shows a flowchart of steps of a method for drug recommendation according to an embodiment of the present disclosure;

FIG. 2B shows a flowchart of an example of step 204 in the method for drug recommendation of FIG. 2A;

FIG. 2C shows a flowchart of an example of step 207 in the method for drug recommendation of FIG. 2A;

FIG. 2D shows a flowchart of an example of step 208 in the method for drug recommendation of FIG. 2A;

FIG. 2E shows a flowchart of an example of step 209 in the method for drug recommendation of FIG. 2A;

FIG. 3 shows a schematic structural diagram of a device for drug recommendation according to an embodiment of the present disclosure;

FIG. 4 shows a schematic structural diagram of a device for drug recommendation according to an embodiment of the present disclosure;

FIG. 5 shows a schematic block diagram of an electronic device according to an embodiment of the present disclosure;

FIG. 6 shows a schematic diagram of an application scenario of a method for drug recommendation according to an embodiment of the present disclosure.

### DETAILED DESCRIPTION

In order to make the above objectives, features and advantages of the present disclosure more obvious and understandable, the present disclosure will be further described in details below with reference to the accompanying drawings and specific implementations.

A general method for drug recommendation tends to recommend commonly used drugs to users, but long-term use of the drugs may result in a problem of poorer effects of normal doses of drugs, or even make the drug completely ineffective, thereby causing difficulties in disease treatment and subject to spread the disease. The embodiments of the present disclosure provide a method and device for drug recommendation. Based on a text semantic similarity between information of a first drug and information of a second drug, a drug can be recommended, and an alternative drug can be recommended to a patient according to the patient's previous medication status, avoiding poorer treatment effects due to long-term use of a same drug.

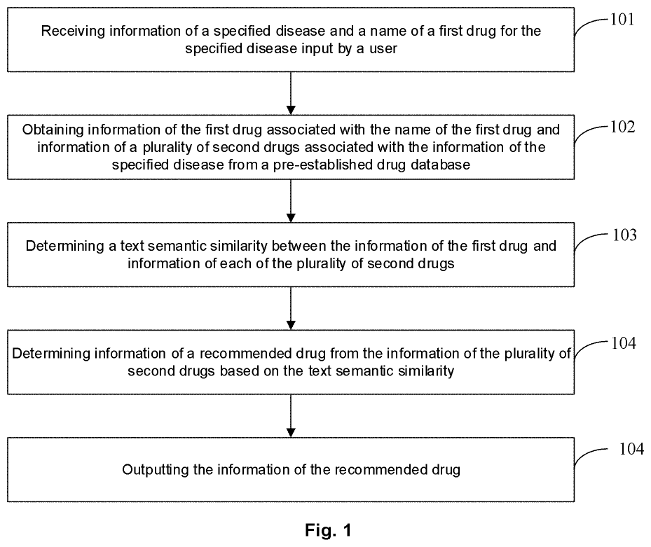

FIG. 1 shows a flowchart of steps of a method for drug recommendation according to an embodiment of the present disclosure.

At step 101, information of a specified disease and a name of a first drug for the specified disease input by a user are received.

The embodiment of the present disclosure aims to provide a system for drug recommendation (for example, the system can be implemented in a server). In response to a drug being intended to be recommended to a user by the system for drug recommendation, for example, the user can click an APP (Application) for the system for drug recommendation on a client, or input an URL for the system for drug recommendation to log in to the system for drug recommendation in a manner of a webpage.

The specified disease refers to a disease input into the system for drug recommendation by the user for searching for a drug for treating the specified disease. The specified disease can be a cold, fever, etc.

It can be understood that the specified disease may be a current disease of the user, or a disease of other users input by the user. Specifically, the specified disease can be determined according to an actual situation, and is not limited in the embodiment of the present disclosure.

The name of the first drug (also called first drug name) may be a name of a common drug previously used by the user or other users to treat the specified disease. For example, in a treatment of a cold, the user's previous common drugs are xx Cold Capsule, xx Gankang Tablet, etc., and then the user may input xx Cold Capsule and xx Gankang Tablet into the system for drug recommendation as the first drug name.

The first drug name for the specified disease input by the user can be a name of one drug, or names of two or more drugs. Specifically, the name of the first drug can be determined according to an actual situation, and is not limited in the embodiment of the present disclosure.

The user can input the name of the specified disease and the first drug name for the specified disease by using a specified interface for the system for drug recommendation presented by the client. Furthermore, in a subsequent process, the system for drug recommendation may perform drug recommendation according to the name of the specified disease and the first drug name for the specified disease input by the user. This will be described in details in the following steps, and will not be repeated here.

After the specified disease and the first drug name for the specified disease input by the user are received, step 102 is performed.

At step 102, information of the first drug associated with the name of the first drug and information of a plurality of second drugs associated with the information of the specified disease are obtained from a pre-established drug database.

The information of the first drug (also called first drug information) may be drug information associated with the first drug name. The first drug information may include, but is not limited to, efficacy information, chemical composition information, expiration information, etc. of the first drug. Specifically, the information of the first drug can be determined according to an actual situation, and is not limited in the embodiment of the present disclosure.

The information of the second drug (also called second drug information) may be information of a drug capable of treating the specified disease other than the first drug. For example, both chemical compositions a and b can treat a specified disease A, and in response to the first drug information comprising the chemical composition a, the chemical composition b may be used as the second drug information.

The drug database refers to a pre-established drug database associated with the system for drug recommendation. An establishment of the drug database may include: crawling diseases and drug information data from a drug information source (for example, a specified drug website or a drug introduction webpage), to establish the drug database.

The detailed process of establishing a drug database will be described in details in the following embodiments, and will not be described in details here.

After the system for drug recommendation receives the specified disease and the first drug name for the specified disease input by the user, the first drug information of the first drug name and the information of the plurality of second drugs associated with the specified disease may be obtained from the pre-established drug database.

The detailed obtaining method for the first drug information and the second drug information will also be described in details in the second embodiment below, and is not limited in the embodiment of the present disclosure.

After the first drug information associated with the first drug name and information of a plurality of second drugs associated with the information of the specified disease are obtained from a pre-established drug database, step 103 is performed.

At step 103, a text semantic similarity between the information of the first drug and information of each of the plurality of second drugs is determined.

The text semantic similarity may be a degree of similarity in semantic between the text of the first drug information and the text of information for each second drug. The text semantic similarity may include an efficacy similarity and an composition similarity, the efficacy similarity may be a similarity between the efficacy information included in the first drug information and the efficacy information included in the second drug information, and the composition similarity may be a similarity between the chemical composition information included in the first drug information and the chemical composition information included in the second drug information.

The obtaining process of the text semantic similarity may include, but is not limited to: processing word segmentation and keyword recognition according to the efficacy information, then obtaining an efficacy information word vector according to a recognized keyword, and calculating the efficacy similarity based on a word vector for the efficacy information of the first drug information and a word vector for the efficacy information of the second drug information. The calculation process of the composition similarity is similar to the above calculation process of the efficacy similarity.

After the efficacy similarity and the composition similarity are obtained, the text semantic similarity may be calculated based on the efficacy similarity and the composition similarity.

The calculation methods of the efficacy similarity, the composition similarity and the text semantic similarity will be described in details in the following embodiments, and will not be repeated here.

After the text semantic similarity between the first drug information and information for each second drug is determined, step 104 is performed.

At step 104, information of a recommended drug is determined from the information of the plurality of second drugs based on the text semantic similarity.

The information of the recommended drug may be drug information used to recommend to users. The information of the recommended drug may include a name of the recommended drug, efficacy information of the recommended drug, and chemical composition information of the recommended drug. Specifically, the information of the recommended drug can be determined according to service demands, and is not limited in the embodiment of the present disclosure.

After the text semantic similarity between the first drug information and information for each second drug is determined, the information of the recommended drug may be obtained from the second drug information. The detailed process of how to obtain the information of the recommended drug from the second drug information will be described in details in the following embodiments, and is not limited in the embodiment of the present disclosure.

After the information of the recommended drug is determined from the second drug information based on each text semantic similarity, step 105 is performed.

At step 105, the information of the recommended drug is output.

After the system for drug recommendation determines the information of the recommended drug, the information of the recommended drug may be output. For example, the information of the recommended drug may be output in form of a pop-up window in a specified interface for the APP for the system for drug recommendation presented by the client. For example, a window is popped-up, displaying the name of the recommended drug, the efficacy information of the recommended drug, and the chemical composition information of the recommended drug, etc.

The information of the recommended drug may also be output in form of voice broadcast, i.e., broadcasting the name of the recommended drug, the efficacy information of the recommended drug, the chemical composition information of the recommended drug, etc. by using voice broadcast.

In a specific implementation, the information of the recommended drug may be output in other manners. For example, the information of the recommended drug may be output in a manner of a combination of the above two outputting methods, etc. The method can be determined according to service demands, and is not limited in the embodiment of the present disclosure.

The embodiment of the present disclosure combines a user's symptom with a commonly used drug to obtain the text semantic similarity between the commonly used drug and other drugs for treating the symptom, such that a recommended alternative drug can be obtained according to the patient's previous medication status.

In the method for drug recommendation provided by the embodiment of the present disclosure, by receiving the specified disease and the first drug name for the specified disease input by the user, the first drug information of the first drug name and the information of the plurality of second drugs associated with the information of the specified disease may be obtained from the pre-established drug database, and the text semantic similarity between the first drug information and information for each second drug may be determined. Further, based on the text semantic similarity, information of the recommended drug may be determined from the information of the plurality of second drugs, and the information of the recommended drug may be output. The embodiment of the present disclosure combines the user's symptom with a commonly used drug to obtain the text semantic similarity between the commonly used drug and other drugs for treating the symptom, a recommended alternative drug can be obtained according to the patient's previous medication status, allowing scientific choosing and purchasing of the patient, and avoiding blind drug usage affecting treatment effects.

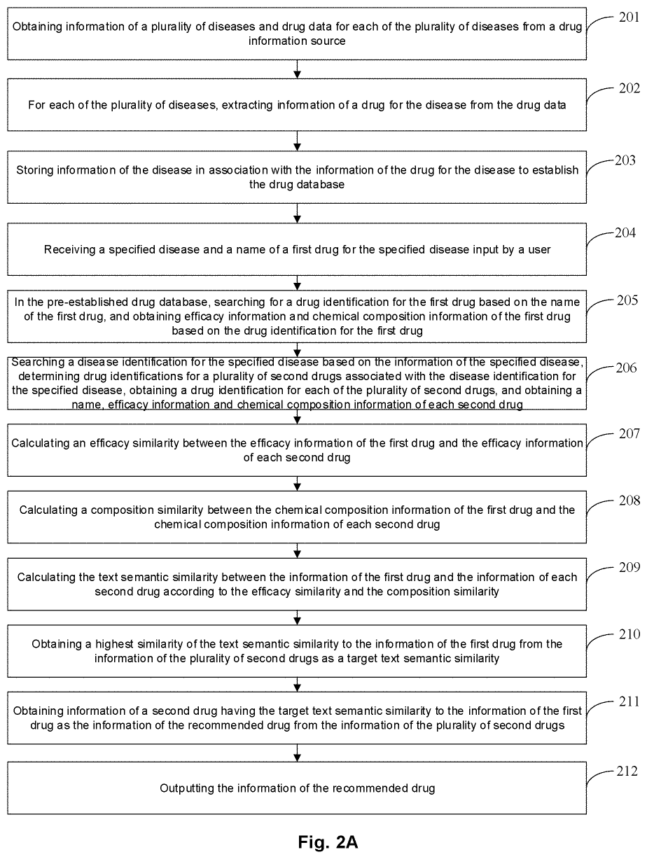

FIG. 2 shows a flowchart of steps of a method for drug recommendation according to an embodiment of the present disclosure.

At step 201, information of a plurality of diseases and drug information of each of the plurality of diseases are obtained from a drug information source.

The drug information source includes a source capable of obtaining information of various diseases and drug information for treating each disease, such as various drug websites, etc.

A plurality of diseases is recorded in the drug information source, such as a cold, fever, dysentery, etc. A drug for the disease may include a drug for treating the disease. For example, drugs for a cold include drugs called xx Cold Capsules, xx Cold Particles, etc.

In the present disclosure, a plurality of diseases and drug information of each disease can be obtained from a drug information source in a manner of web crawlers, such as crawling drug-related data from major drug websites. In this step, in order to obtain a knowledge base as comprehensive as possible, the number of drug data sources can be as much as possible.

After the plurality of diseases and the drug information of the diseases are obtained at the drug information source end, step 202 is performed.

At step 202, for each of the plurality of diseases, information of the drug for the disease is extracted from the drug data. The information of the drug includes, but is not limited to, a name of the drug, efficacy information of the drug, and chemical composition information of the drug.

After the plurality of diseases and the drug information of each of the plurality of diseases are obtained from the drug information source, the name, efficacy information and chemical composition information of the drug may be extracted from the drug information used for treating each disease. For example, the name of the drug is xxx Cold Particles, the efficacy information is heat-clearing and detoxifying, wind-heat and cold, fever, sore throat, and thirst, and the chemical composition information is acetaminophen, pseudoephedrine hydrochloride, etc.

The extraction process can be obtaining all data information of each drug webpage by using a requests framework in Python, parsing the webpage by using a lxml module to extract useful information (i.e., the efficacy and chemical compositions of each drug) specifically, and storing the useful information as drug information with the name of the drug as a txt name under a disease folder.

In some embodiments, the name, efficacy information, and chemical composition information of the drug in the drug information can also be extracted in other methods and can be determined according to service demands, and is not limited in the embodiment of the present disclosure.

After the name, efficacy information and chemical composition information of the drug is extracted, step 203 is performed.

At step 203, information of the disease is stored in association with the information of the drug for the disease to establish the drug database.

For example, the information of the drug may include the name of the drug, the efficacy information of the drug, and the chemical composition information of the drug. After extracting the name, efficacy information, and chemical composition information of the drug from the drug data, a drug database may be established based on the disease, the name, efficacy information, and chemical composition of the drug, as well as the association relationship among them, for example, as shown in Table 1 below:

TABLE 1

| | | | Chemical Composition |
|---|---|---|---|
| Disease | Name of Drug | Efficacy Information | information |
| M | A | a, b, c | 1, 2, 3 |
| N | B | b, d | 2, 4 |

As shown in Table 1, the name of the drug for treating Disease M is A, the efficacy information of Drug A is a, b, and c, the chemical composition information of Drug A is 1, 2, 3; the name of the drug for treating disease N is B, the efficacy information of Drug B is b and d, and the chemical composition information of Drug B is 2 and 4.

It can be understood that the above examples are merely examples for better understanding of the technical solutions of the embodiments of the present disclosure, and are not intended as the only limitation on the embodiments of the present disclosure.

After the drug database is established based on the disease, the name, efficacy information, and chemical composition information of the drug, as well as the association relationship among them, step 204 is performed.

At step 204, a specified disease and a name of a first drug for the specified disease input by a user are received.

The embodiment of the present disclosure aims to provide a system for drug recommendation. In response to a drug being intended to be recommended to a user by the system for drug recommendation, the user can click an APP (Application) for the system for drug recommendation on a client, or input an URL for the system for drug recommendation to log in to the system for drug recommendation in a manner of a webpage.

The specified disease may be a disease input into the system for drug recommendation by the user for searching for a drug for treating the specified disease. The specified disease can be a cold, fever, etc.

It can be understood that the specified disease may be a current disease of the user, or a disease of other users input by the user. Specifically, the specified disease can be determined according to an actual situation, and is not limited in the embodiment of the present disclosure.

The first drug name may include a name of a common drug previously used by the user or other users to treat the specified disease. For example, in a treatment of a cold, the user's previous common drugs are xx Cold Capsule, xx Gankang Tablet, etc., and then the user may input xx Cold Capsule and xx Gankang Tablet into the system for drug recommendation as the first drug name.

The first drug name for the specified disease input by the user can be a name of one drug, or names of two or more drugs. Specifically, the first drug name can be determined according to an actual situation, and is not limited in the embodiment of the present disclosure.

The user can input the specified disease and the first drug name for the specified disease by using a specified interface for the system for drug recommendation presented by the client, such that in a subsequent process, the system for drug recommendation may perform drug recommendation according to the specified disease and the first drug name for the specified disease input by the user. This will be described in details in the following steps, and will not be repeated here.

Alternatively, in the establishment process of the above drug database, the drug database may be established based on a disease identification of each disease and a drug identification of each drug, and will be described in details in the following specific implementation.

In a specific implementation of the embodiment of the present disclosure, as shown in FIG. 2B, the above step 204 may include sub-steps A1 to A3.

At sub-step A1, a disease identification of the disease for the information of the disease is set, and a drug identification of each drug for a name of each drug for the disease is set.

In an embodiment of the present disclosure, disease identifications of different diseases may be preset for the names of the diseases. For example, a disease identification of Disease M is Xm, and a disease identification of Disease N is Xn, etc.

A drug identification of each drug may also be preset for a name of each drug for the disease. For example, a drug identification of Drug A is I, and a drug identification of Drug B is II, etc.

It can be understood that the disease identification and the drug identification may be digital identifications, English character identifications, or other forms of identifications. Specifically, the disease identification and the drug identification can be determined according to service demands.

In the present disclosure, in order to distinguish between a drug and a disease, the disease identification and the drug identification may be set in different forms. For example, in case that the disease identification being in form of a digital identification, the drug identification may be in form of an English character identification; while in case that the disease identification being in form of an English character identification, the drug identification may be in form of a digital identification.

It can be understood that the above examples are merely examples for better understanding of the technical solutions of the embodiments of the present disclosure, and are not intended as the only limitation on the embodiments of the present disclosure.

After the disease identification of the disease and the drug identification of each drug are set, sub-step A2 is performed.

At sub-step A2, a mapping table between the disease identification of the disease and the drug identification of each drug for the disease are established.

After setting the disease identification of the disease and the drug name identification of the name of each drug, an association relationship between the disease identification and each drug identification can be established. For example, a disease identification for Disease M is Xm, and names of drugs treating Disease M are A, B and C, a drug identification of Drug A is I, a drug identification of Drug B is II, and a drug identification of Drug C is III, then a mapping table between the disease identification Xm and drug identifications I, II and III can be established.

After the association relationship between the disease identification and each drug identification is established, sub-step A3 is performed.

At sub-step A3, the drug identification of each drug in the mapping table and the efficacy information and the chemical composition information of the drug are stored in form of a dictionary.

After establishing the mapping table (association relationship) between the disease identification and each drug identification, the drug identification for each drug in the mapping table and the efficacy information and chemical composition information of the drug may be stored in the form of the dictionary, such as a CSV (Comma-Separated Values) file storing data in form of plain text, etc., thereby establishing a drug database.

The above storing method provided by the embodiment of the present disclosure can perform data cleaning and avoid redundancy. For example, data not included in the drug database may be added, and for repeated data of a same drug, a more detailed description may be selected for storage, and organized into a CSV file in the form of the dictionary. For example, an ID number may be set for each disease, and an ID number may be set for each drug to avoid redundancy, and to improve search efficiency. Searching for a number is much more efficient than searching for a text, and the number can be converted into text for output.

After the information of the specified disease and the first drug name for the specified disease input by the user are received, step 205 is performed.

At step 205, in the pre-established drug database, a drug identification of the first drug is searched based on the first drug name, and efficacy information and chemical composition information of the first drug are obtained based on the drug identification of the first drug.

In the drug database, each drug has a unique drug identification specific to each drug. The drug identification may be a digital identification, for example, a drug identification of Drug A is I, a drug identification of Drug B is II, etc. The drug identification may be an English character identification, for example, a drug identification of Drug C is Yc, and a drug identification of Drug D is Yd. Specifically, the drug identification may be determined according to an actual situation, and is not limited in the embodiment of the present disclosure.

It can be understood that the forms of the drug identifications of all drugs are consistent. For example, the drug identifications for all drugs are digital identifications, or the drug identifications of all drugs are English character identifications, etc.

The drug identification of the first drug (also called first drug identification) may be a drug identification associated with the first drug name.

The efficacy information of the first drug (also called first efficacy information) may be efficacy information associated with the identification (or name) of the first drug.

The chemical composition information of the first drug (also called first chemical composition information) may be chemical composition information associated with the drug identification (or name) for the first drug.

Since the first drug name and the first drug identification associated with the first drug are pre-stored in the drug database, the first drug identification of the first drug name can be searched in the pre-established drug database, and according to the association relationship between the first drug identification (or the first drug name) and the efficacy information and chemical composition information of the first drug identification (or the first drug name), the first efficacy information and the first chemical composition information associated with the first drug identification are obtained from the drug database.

In the pre-established drug database, after the first drug identification associated with the first drug name is searched, and the efficacy information and chemical composition information associated with the first drug identification is obtained, step 206 is performed.

At step 206, a disease identification of the specified disease is searched based on the information of the specified disease, drug identifications of a plurality of second drugs associated with the disease identification of the specified disease are determined, a drug identification of each of the plurality of second drugs is obtained, and a name, efficacy information and chemical composition information of each second drug are obtained.

The disease identification of the disease may be a disease identification associated with the information of the disease.

The drug identification of the second drug (also called second drug identification) may be a drug identification for a drug capable of treating the specified disease other than the first drug.

After the disease identification is determined, the drug identification of the second drug may be determined according to an association relationship between the disease identification and the drug identification in the drug database.

The name of the second drug (also called second drug name) may be a name of a drug associated with the second drug identification.

The efficacy information of the second drug (also called second efficacy information) may be efficacy information of the drug associated with the second drug identification.

The chemical composition information of the second drug (also called second chemical composition information) may be chemical composition information of the drug associated with the second drug identification.

According to an association relationship between the second drug identification and the efficacy information and the chemical composition information associated with the second drug name, the second efficacy information and the second chemical composition information associated with the second drug identification are obtained from the drug database.

After the disease identification of the specified disease is searched, the drug identifications of the plurality of second drugs associated with the disease identification are determined, and the names, efficacy information and chemical composition information of the second drugs associated with the drug identifications of the plurality of second drugs are obtained, step 207 is performed.

At step 207, an efficacy similarity between the efficacy information of the first drug and the efficacy information of each second drug is calculated.

The efficacy similarity may be a similarity between the efficacy information associated with the drug identification of the first drug and the efficacy information associated with the drug identification of the second drug.

After the efficacy information of the first drug and the efficacy information of each second drug are obtained, the efficacy similarity between the efficacy information of the first drug and the efficacy information of each second drug may be calculated, and is described in details in the following specific implementation.

In a specific implementation of the embodiment of the present disclosure, the efficacy information of the first drug includes text describing efficacy of the first drug, and the efficacy information of the second drug includes text describing efficacy of the second drug. As shown in FIG. 2C, the above step 207 may include sub-steps B1 to B3.

At sub-step B1, word segmentation and keyword recognition processing are performed on the text describing the efficacy information of the first drug to generate a first word vector sequence of a first keyword for the efficacy information of the first drug.

In an embodiment of the present disclosure, after the efficacy information of the first drug is obtained, word segmentation may be performed on the text for the efficacy information of the first drug. For example, after word segmentation for the efficacy of a fever drug xx Cold Tablets, following words may be obtained: febrifuge, cold, fever, pharyngalgia, and thirst.

In the present disclosure, the word segmentation may be processed in a manner of jieba word segmentation. In a specific implementation, other manners of word segmentation may also be used. Specifically, the manner of word segmentation may be determined according to service demands, and is not limited by the embodiment of the present disclosure.

The first keyword may include a keyword extracted from the text included in the first efficacy information after word segmentation and keyword recognition processing.

After the text for the first efficacy information is segmented, keyword recognition processing may be performed on each piece of segmentation, such as using CRF Algorithm (Conditional Random Field Algorithm) for keyword recognition. For example, after keyword extraction for the text included in the efficacy information of the fever drug xx Cold Tablets, the extracted keywords are febrifuge, cold, fever, pharyngalgia, and thirst.

It can be understood that the above examples are merely examples for better understanding of the technical solutions of the embodiment of the present disclosure, and are not intended as the only limitation on the embodiments of the present disclosure.

In performing word segmentation and keyword recognition processing on the text included in the first efficacy information, one or more first keywords may be extracted, and a first word vector sequence may be generated based on the one or more first keywords. The method of generating the word vector sequence based on the keywords is not limited by the embodiment of the present disclosure and will not be described in details here.

At sub-step B2, word segmentation and keyword recognition processing are performed on text describing efficacy information of each second drug to generate a second word vector sequence of a second keyword for the efficacy information of each second drug.

After the efficacy information of the second drug is obtained, word segmentation may be performed on the text for the second efficacy information. For example, after word segmentation for the efficacy of a fever drug xx Cold Tablets, each piece of segmentation may be obtained: febrifuge, cold, fever, pharyngalgia, and thirst.

In the present disclosure, the word segmentation processing may in a manner of jieba word segmentation. In a specific implementation, other manners of word segmentation may also be used. The manner of word segmentation may be determined according to service demands, and is not limited by the embodiment of the present disclosure.

The second keyword may include a keyword extracted by performing word segmentation and keyword recognition processing on the text included in the efficacy information of the second data.

After the text for the second efficacy information is segmented, keyword recognition processing may be performed on each piece of segmentation, such as using CRF Algorithm (Conditional Random Field Algorithm) for keyword recognition. For example, after keyword extraction for the text included in the efficacy information of the fever drug xx Cold Tablets, the extracted keywords are febrifuge, cold, fever, pharyngalgia, and thirst.

It can be understood that the above examples are merely examples for better understanding of the technical solutions of the embodiments of the present disclosure, and are not intended as the only limitation on the embodiments of the present disclosure.

In performing word segmentation and keyword recognition processing on the text included in the second efficacy information, one or more second keywords may be extracted, and a second word vector sequence may be generated based on the one or more second keywords. The method of generating the word vector sequence based on the keywords is not limited by the embodiment of the present disclosure and will not be described in details here.

At sub-step B3, the first word vector sequence and the second word vector sequence are input into a first neural network model, to calculate the efficacy similarity. The efficacy similarity may be proportional to a degree of similarity of the efficacy information.

The first neural network model may be a neural network model used to calculate the efficacy similarity according to a word vector sequence for efficacy information.

The first neural network model is obtained by training according to word vector sequence samples for the efficacy information. The training process of the first neural network model is not limited in the embodiment of the present disclosure, and will not be described in details here.

After obtaining the first word vector sequence for the first efficacy information and the second word vector sequence for information of each second efficacy, the first word vector sequence and each second word vector sequence may be input into the first neural network model.

Furthermore, the first neural network model may output the efficacy similarity between the first efficacy information and information of each second efficacy.

The efficacy similarity output by the first neural network model may be proportional to the degree of the similarity of the efficacy information, i.e., the higher the similarity between the first efficacy information and the second efficacy information, the higher the value of the efficacy similarity output by the first neural network model. On the contrary, the lower the similarity between the first efficacy information and the second efficacy information, the lower the value of the efficacy similarity output by the first neural network model. It can be understood that the above specific implementation is only a solution for calculating the efficacy similarity for a better understanding of the technical solutions of the embodiments of the present disclosure. In practical applications, those skilled in the art may also use other methods to calculate the efficacy similarity between the first efficacy information and information of each second efficacy.

At step 208, a composition similarity between the chemical composition information of the first drug and the chemical composition information of each second drug is calculated.

The composition similarity may be a similarity between the first chemical composition information associated with the first drug identification and the second chemical composition information associated with the second drug identification.

After the first chemical composition information and information of each second chemical composition are obtained, the efficacy similarity between the first chemical composition information and information of each second chemical composition may be calculated, and will be described in details in the following specific implementation.

In a specific implementation of the embodiment of the present disclosure, the chemical composition information of the first drug includes text describing chemical composition of the first drug, and the chemical composition information of the second drug includes text describing chemical composition of the second drug. As shown in FIG. 2D, the above step 208 may include sub-steps C1 to C3.

At sub-step C1, word segmentation and keyword recognition processing are performed on the text describing the chemical composition of the first drug to generate a third word vector sequence of a third keyword for the chemical composition information of the first drug.

In an embodiment of the present disclosure, after the first chemical composition information is obtained, word segmentation may be performed on the text included in the first chemical composition information. For example, after word segmentation for the chemical composition information of a fever drug xx Cold Tablets, following words may be obtained: acetaminophen and pseudoephedrine hydrochloride.

In the present disclosure, the word segmentation may be implemented by split in Python, i.e., the word segmentation is performed according to the punctuation of the chemical composition. In a specific implementation, other manners of word segmentation may also be used. Specifically, the manner of word segmentation may be determined according to service demands, and is not limited by the embodiment of the present disclosure.

The third keyword may include a keyword extracted from the text included in the first chemical composition information after word segmentation and keyword recognition processing.

After the text included in the first chemical composition information is segmented, keyword recognition processing may be performed on each piece of segmentation, such as using CRF Algorithm (Conditional Random Field Algorithm) for keyword recognition.

In performing word segmentation and keyword recognition processing on the text included in the first chemical composition information, one or more third keywords are extracted, and the third word vector sequence can be generated based on the one or more third keywords, such as using word2vec to convert the word elements of the third keywords into a format of word vectors. The method of generating the word vector sequence based on the keywords is not limited in the embodiment of the present disclosure and will not be described in details here.

At sub-step C2, word segmentation and keyword recognition processing are performed on text describing the chemical composition of each second drug to generate a fourth word vector sequence of a fourth keyword for the chemical composition information of the second drug.

After the first chemical composition information is obtained, word segmentation may be performed on the text included in the second chemical composition information. For example, after word segmentation for the chemical composition information of a fever drug xx Cold Tablets, following words may be obtained: acetaminophen and pseudoephedrine hydrochloride.

In the present disclosure, the word segmentation may be implemented by split in Python, i.e., the word segmentation is performed according to the punctuation of the chemical composition. In a specific implementation, other manners of word segmentation may also be used. The manner of word segmentation may be determined according to service demands, and is not limited by the embodiment of the present disclosure.

The fourth keyword may include a keyword extracted from the text included in the second chemical composition information after word segmentation and keyword recognition processing.

After the text included in the second chemical composition information is segmented, keyword recognition processing may be performed on each piece of segmentation, such as using CRF Algorithm (Conditional Random Field Algorithm) for keyword recognition.

In performing word segmentation and keyword recognition processing on the text included in the second chemical composition information, one or more fourth keywords can be extracted, and the fourth word vector sequence can be generated based on the one or more fourth keywords, such as using word2vec to convert the word elements of the fourth keywords into a format of word vectors. The method of generating the word vector sequence based on the keywords is not limited in the embodiment of the present disclosure and will not be described in details here.

At sub-step C3, the third word vector sequence and the fourth word vector sequence are input into a second neural network model, to calculate composition similarity. The composition similarity may be inversely proportional to a similarity of the chemical composition information.

The second neural network model may be a neural network model used to calculate the composition similarity according to a word vector sequence for chemical composition information.

The second neural network model is obtained by training according to word vector sequence samples for the chemical composition information. The training process of the second neural network model is not limited in the embodiment of the present disclosure, and will not be described in details here.

After obtaining the third word vector sequence for the first chemical composition information and the fourth word vector sequence for information of each second chemical composition, the third word vector sequence and each fourth word vector sequence may be input into the second neural network model.

Furthermore, the second neural network model may output the composition similarity between the first chemical composition information and information of each second chemical composition.

The composition similarity output by the second neural network model may be inversely proportional to the similarity of the chemical composition information, i.e., the higher the similarity between the first chemical composition information and the second chemical composition information, the lower the value of the composition similarity output by the second neural network. On the contrary, the lower the similarity between the first chemical composition information and the second chemical composition information, the higher the value of the composition similarity output by the second neural network model.

It can be understood that the above specific implementation is only a solution for calculating the composition similarity for a better understanding of the technical solutions of the embodiments of the present disclosure. In practical applications, those skilled in the art may also use other methods to calculate the composition similarity between the first chemical composition information and information of each second chemical composition.

After the efficacy similarity and the composition similarity are calculated, step 209 is performed.

At step 209, the text semantic similarity between the information of the first drug and the information of each second drug is calculated according to the efficacy similarity and the composition similarity.

After the efficacy similarity between the first efficacy information and information of each second efficacy and the composition similarity between the first chemical composition information and information of each second chemical composition is calculated, the text semantic similarity between each first drug information and information for each second drug can be calculated based on the efficacy similarity and each composition similarity.

The specific calculation process of text semantic similarity may be described in details with reference to the following specific implementation.

In a specific implementation of the embodiment of the present disclosure, as shown in FIG. 2E, the above step 209 may include sub-steps D1 and D2.

At sub-step D1, for each second drug, an average value of the efficacy similarity and the composition similarity to the first drug is calculated.

In an embodiment of the present disclosure, the average value refers to an average value of efficacy similarity and composition similarity between information of each second drug and the first drug information. For example, the first drug includes Drug A, the second drug includes Drug B and Drug C. An efficacy similarity between Drug A and Drug B is 0.8, and a composition similarity between Drug A and Drug B is 0.6. An average similarity between Drug A and Drug B is (0.8+0.6)/2=0.7. An efficacy similarity between Drug A and Drug C is 0.7, and a composition similarity between Drug A and Drug C is 0.5. An average similarity between Drug A and Drug C is (0.7+0.5)/2=0.6.

It can be understood that the above examples are merely examples for better understanding of the technical solutions of the embodiments of the present disclosure, and are not intended as the only limitation on the embodiments of the present disclosure.

After the average value of the efficacy similarity and the composition similarity between information of each second drug and the first drug information is calculated, sub-step D2 is performed.

At sub-step D2, the average value is determined as the text semantic similarity between the information of the first drug and the information of each second drug.

After the average value of the efficacy similarity and the composition similarity between information for each second drug and the first drug information is calculated, the average value may be used as the text semantic similarity between information for each second drug and the first drug information.

In the above process, since the efficacy similarity is proportional to the similarity of the efficacy information, while the composition similarity is inversely proportional to the similarity of the chemical composition information, the higher the average value of the efficacy similarity and the composition similarity is, the higher the degree of similarity to the specified disease is indicated, and the lower the degree of similarity to chemical composition.

In an embodiment of the present disclosure, by calculating the average value of the efficacy similarity and the composition similarity of information for each second drug, the average values can be sorted in descending order, the average value with the higher order has a higher degree of similarity to the specified disease is indicated, and has a lower degree of similarity to chemical composition.

The higher the degree of efficacy similarity between the recommended drugs and the user's commonly used drugs is, the closer the effect of treating the specified disease is to the user's commonly used drugs, while the lower the chemical composition similarity is, the lower the resistance of the user with the specified disease is to the recommended drugs. That is, the embodiment of the present disclosure is intended to select drugs with less resistance to users and with a treatment effect closer to the commonly used drugs as recommended drugs, avoiding the problem of poor effects caused by high drug resistance in recommending drugs to users, and providing users with recommended drugs with better curative effects to improve the treatment effects.

At step 210, a highest similarity of the text semantic similarities to the information of the first drug is obtained from the information of the plurality of second drugs as a target text semantic similarity.

The target text semantic similarity refers to a text semantic similarity with a highest similarity value of the text semantic similarities for information for each second drug, and the second drug information for the target text semantic similarity is drug information of the second drug information having the highest degree of efficacy similarity and the lowest degree of chemical composition similarity to the first drug information of the specified disease.

After the target text semantic similarity with the highest similarity value of the text semantic similarities is obtained, step 211 is performed.

At step 211, information of a second drug having the target text semantic similarity to the information of the first drug (also called second target drug information) is obtained as the information of the recommended drug from the information of the plurality of second drugs.

The second target drug information may be second drug information of the target text semantic similarity among information for each second drug.

After the target text semantic similarity with the highest similarity value of the text semantic similarities is obtained, the second target drug information having the target text semantic similarity can be obtained from information for each second drug according to the text semantic similarity, and the second target drug information is set as the information of the recommended drug.

After the second target drug information having the target text semantic similarity is obtained and the second target drug information is set as the information of the recommended drug, step 212 is performed.

At step 212, the information of the recommended drug is output.

After the drug recommendation server determines the information of the recommended drug, the information of the recommended drug may be output to the client. The client may present the recommended drug information in form of a pop-up window in a specific interface for the APP for the system for drug recommendation. For example, a window is popped-up, displaying the name of the recommended drug, the efficacy information of the recommended drug, and the chemical composition information of the recommended drug, etc.

The method of outputting the information of the recommended drug may also be outputting the information of the recommended drug in form of voice broadcast, i.e., broadcasting the name of the recommended drug, the efficacy information of the recommended drug, the chemical composition information of the recommended drug, etc. by using voice broadcast.

In a specific implementation, the information of the recommended drug may be output in other manners. For example, the information of the recommended drug may be output in a manner of a combination of the above two outputting methods, etc. Specifically, the method can be determined according to service demands, and is not limited in the embodiment of the present disclosure.

The embodiment of the present disclosure combines a user's symptom with a commonly used drug to obtain the text semantic similarity between the commonly used drug and other drugs for treating the symptom, and a recommended alternative drug can be obtained according to the patient's previous medication status.

In the method for drug recommendation provided by the embodiment of the present disclosure, by receiving the specified disease and the first drug name for the specified disease input by the user, the first drug information of the first drug name and the information of the plurality of second drugs associated with the information of the specified disease may be obtained from the pre-established drug database, and the text semantic similarity between the first drug information and information for each second drug may be determined. Further, based on the text semantic similarity, information of the recommended drug may be determined from the information of the plurality of second drugs, and the information of the recommended drug may be output. The embodiment of the present disclosure combines the user's symptom with a commonly used drug to obtain the text semantic similarity between the commonly used drug and other drugs for treating the symptom, a recommended alternative drug can be obtained according to the patient's previous medication status, allowing scientific choosing and purchasing of the patient, and avoiding blind drug usage.

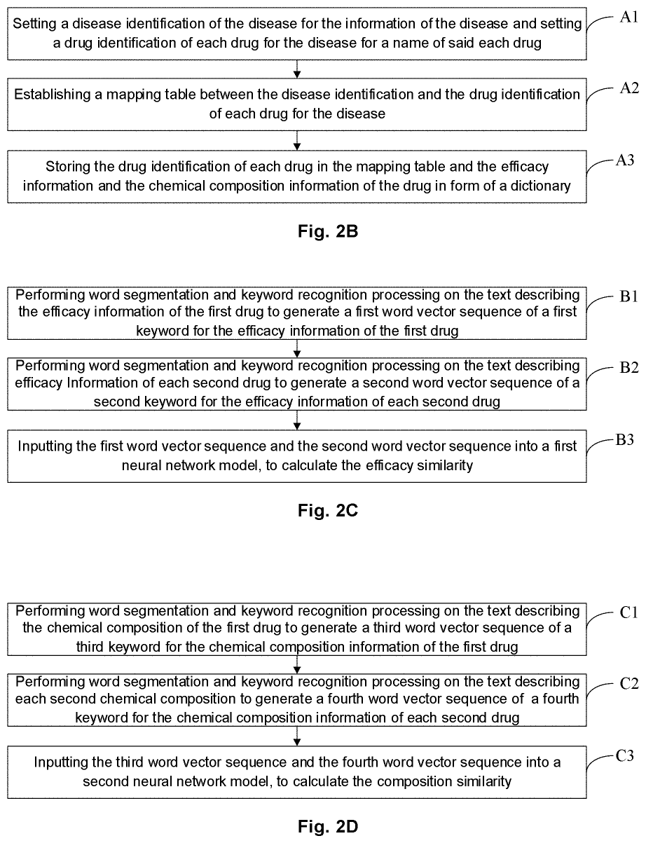

FIG. 3 shows a schematic structural diagram of a device for drug recommendation according to an embodiment of the present disclosure.

The device for drug recommendation may include a disease and drug receiving module 310, a first and second drug obtaining module 320, a similarity determining module 330, a recommended drug determining module 340, and a recommended drug outputting module 350.

The disease and drug receiving module 310 is used to receive a specified disease and a name of a first drug for the specified disease input by a user.

The first and second drug obtaining module 320 is used to obtain first drug information associated with the first drug name and information of a plurality of second drugs associated with the specified disease from a pre-established drug database.

The similarity determining module 330 is used to determine a text semantic similarity the first drug information and information for each second drug.

The recommended drug determining module 340 is used to determine information of a recommended drug from information for each second drug based on the text semantic similarity.

The recommended drug outputting module 350 is used to output the information of the recommended drug.

In the device for drug recommendation provided by the embodiment of the present disclosure, by receiving the specified disease and the first drug name for the specified disease input by the user, the first drug information of the first drug name and the information of the plurality of second drugs associated with the specified disease may be obtained from the pre-established drug database, and the text semantic similarity between the first drug information and information for each second drug may be determined. Further, based on the text semantic similarity, information of the recommended drug may be determined from information for each second drug, and the information of the recommended drug may be output. The embodiment of the present disclosure combines the user's symptom with a commonly used drug to obtain the text semantic similarity between the commonly used drug and other drugs for treating the symptom, a best recommended alternative drug can be obtained according to the patient's previous medication status, allowing scientific choosing and purchasing of the patient, and avoiding blind drug usage to achieve a best treatment effect.

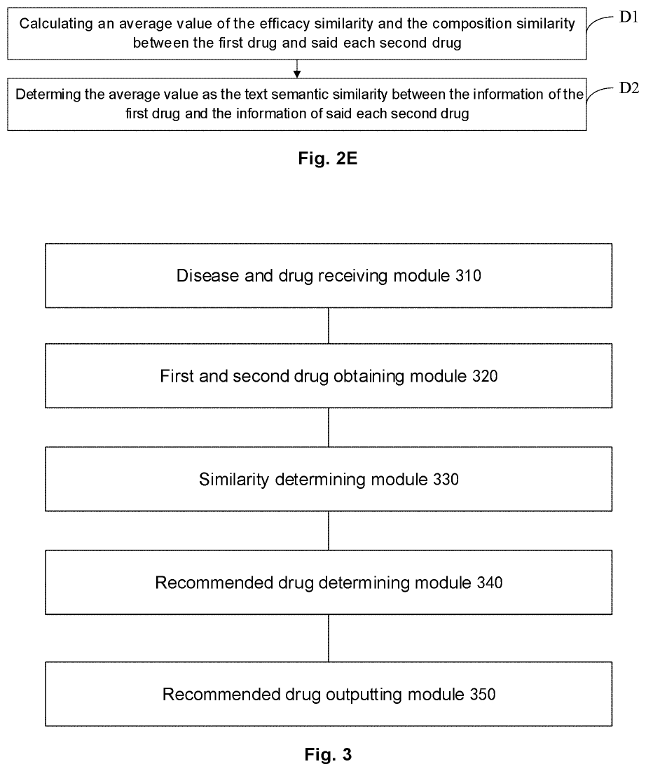

FIG. 4 shows a schematic structural diagram of a device for drug recommendation according to an embodiment of the present disclosure.

The device for drug recommendation may specifically include a drug information obtaining module 410, a drug information extracting module 420, a drug database establishing module 430, a disease and drug receiving module 440, a first and second drug obtaining module 450, a similarity determining module 460, a recommended drug determining module 470 and a recommended drug outputting module 480.

The drug information obtaining module 410 is used to obtain a plurality of diseases and drug information of the diseases from a drug information source end.

The drug information extracting module 420 is used to extract a drug name, efficacy information and chemical composition information of the drug information.

The drug database establishing module 430 is used to establish a drug database according to the disease, the drug name, the efficacy information, the chemical composition information, and the association relationship among them.

The disease and drug receiving module 440 is used to receive a specified disease and a name of a first drug for the specified disease input by a user.

The first and second drug obtaining module 450 is used to obtain first drug information associated with the first drug name and information of a plurality of second drugs associated with the information of the specified disease from a pre-established drug database.

The similarity determining module 460 is used to determine a text semantic similarity between the first drug information and information for each second drug.

The recommended drug determining module 470 is used to determine information of a recommended drug from information for each second drug based on the text semantic similarity.

The recommended drug outputting module 480 is used to output the information of the recommended drug.

For example, the drug database establishing module 430 may include:

an identification setting sub-module for setting a disease identification of the disease and a drug identification for a name of each drug;

an association relationship establishing sub-module for establish an association relationship between the disease identification and the drug identification of each drug; and

a drug database establishing sub-module for storing the association relationship, each efficacy information and each chemical composition information in form of a dictionary to establish a drug database.

For example, the first and second drug obtaining module 450 may include:

a first information searching sub-module 4501 for searching for a first drug identification in the pre-established drug database, and obtain the first efficacy information and the first chemical composition information of the first drug identification information; and

a second information searching sub-module 4502 for searching for the first disease identification of the specified disease, determine a plurality of second drug identifications associated with the first disease identification, and obtain the second drug name, the second efficacy information and the second chemical composition information for each of the plurality of second drug identifications.

For example, the similarity determining module 460 may include:

an efficacy similarity calculating sub-module 4601 for calculating an efficacy similarity between the first efficacy information and information of each second efficacy;

a composition similarity calculating sub-module 4602 for calculating a composition similarity between the first chemical composition information and information of each second chemical composition; and

a similarity calculating sub-module 4603 for calculating the text semantic similarity between each first drug information and information for each second drug according to the efficacy similarity and the composition similarity.

For example, the efficacy similarity calculating sub-module 4601 may include:

a first word vector sequence generating sub-module for performing word segmentation and keyword recognition processing on text included in the first efficacy information, and generate a first word vector sequence for a first keyword for the first efficacy information;

a second word vector sequence generating sub-module for sequentially performing word segmentation and keyword recognition processing on text included in the second efficacy information for information of each second efficacy, and generate a second word vector sequence for a second keyword for the second efficacy information; and

a power similarity obtaining sub-module for inputting the first word vector sequence and the second word vector sequence into a first neural network model and calculate the efficacy similarity; wherein the efficacy similarity is proportional to a degree of similarity of the efficacy information.

For example, the composition similarity calculating sub-module 4602 may include:

a third word vector sequence generating sub-module for performing word segmentation and keyword recognition processing on text included in the first chemical composition information, and generate a third word vector sequence for a third keyword for the first chemical composition information;

a fourth word vector sequence generating sub-module for sequentially performing word segmentation and keyword recognition processing on text included in the second chemical composition information for information of each second chemical composition, and generate a fourth word vector sequence for a fourth keyword for the second chemical composition information; and

a composition similarity obtaining sub-module for inputting the third word vector sequence and the fourth word vector sequence into a second neural network model and calculate the composition similarity; wherein, the composition similarity is inversely proportional to a degree of similarity of chemical composition information.

For example, the similarity calculating sub-module 4603 may include:

an average value calculating sub-module for calculating an average value of the efficacy similarity and the composition similarity of information for each second drug; and

a semantic similarity obtaining sub-module for set the average value of information for each second drug as the text semantic similarity of information for each second drug.

Optionally, the recommended drug determining module 470 includes:

a target similarity obtaining sub-module 4701 for obtain a text semantic similarity with a highest similarity value of each text semantic similarity; and

a recommended drug obtaining sub-module 4702 is configured to obtain second target drug information for the target text semantic similarity, and set the second target drug information as the information of the recommended drug.

In the device for drug recommendation provided by the embodiment of the present disclosure, by receiving the specified disease and the first drug name for the specified disease input by the user, the first drug information of the first drug name and the information of the plurality of second drugs associated with the specified disease may be obtained from the pre-established drug database, and the text semantic similarity between the first drug information and information for each second drug may be determined. Further, based on the text semantic similarity, information of the recommended drug may be determined from information for each second drug, and the information of the recommended drug may be output. The embodiment of the present disclosure combines the user's symptom with a commonly used drug to obtain the text semantic similarity between the commonly used drug and other drugs for treating the symptom, a best recommended alternative drug can be obtained according to the patient's previous medication status, allowing scientific choosing and purchasing of the patient, and avoiding blind drug usage to achieve a best treatment effect.

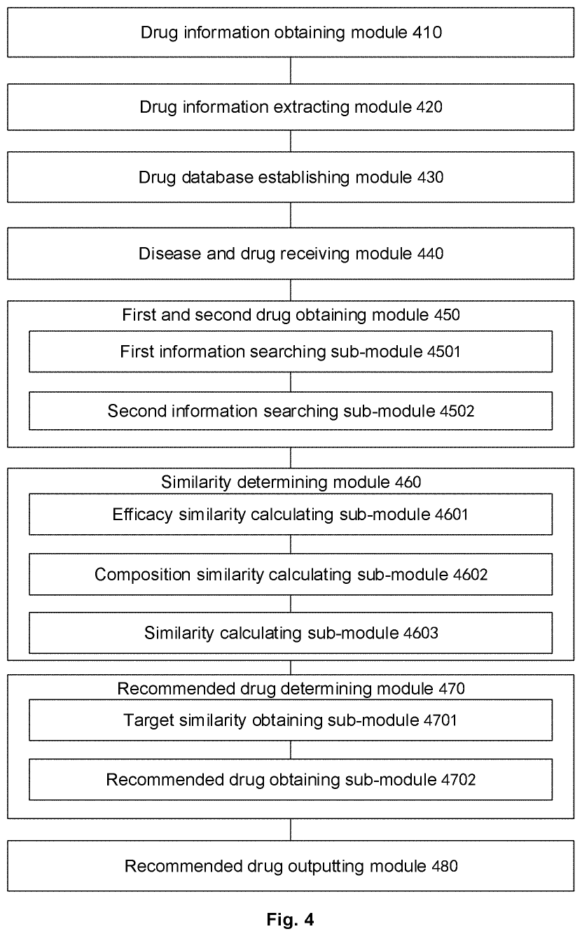

FIG. 5 shows a schematic block diagram of an electronic device according to an embodiment of the present disclosure. As shown in FIG. 5, the electronic device 500 includes a memory 501 and a processor 502.

The memory 501 stores executable instructions for the processor. The processor 502 may execute the instructions to perform the method for drug recommendation of any one of the above embodiments.

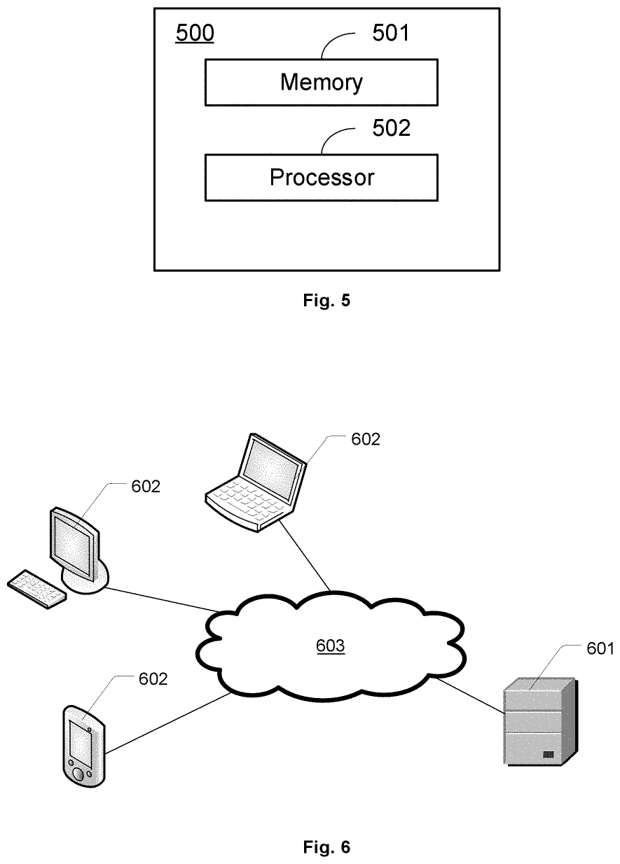

FIG. 6 shows a schematic diagram of an application scenario of a method for drug recommendation according to an embodiment of the present disclosure. As shown in FIG. 6, the application scenario includes a server 601 and at least one client 602, and the server 601 and the client 602 may be connected through a network 603. The method for drug recommendation of an embodiment of the present disclosure may be performed in the server 601. The server 601 may be implemented by the electronic device according to the above embodiment of the present disclosure. The network 603 includes, but is not limited to an Internet, a local area network, a wide area network, etc. Examples of the client 602 include, but are not limited to, a PC, a tablet computer, a notebook computer, etc. The embodiment of the present disclosure is not limited to this, and the method for drug recommendation of the embodiment of the present disclosure can also be applied to other scenarios.

For simple description, the above device embodiments are all expressed as a series of action combinations, but those skilled in the art should know that the present disclosure is not limited by the described sequence of actions, because according to the present disclosure, some steps can be performed in other sequences or performed simultaneously. Furthermore, those skilled in the art should also know that the embodiments described in the description are all preferred embodiments, and the involved actions and modules are not necessarily required by the present disclosure.

The various embodiments in the description are described in a progressive manner. Each embodiment focuses on the differences from other embodiments, and the same or similar parts among the various embodiments can be referred to each other. The various embodiments can be combined.

Finally, it should be noted that in this article, relational terms such as first and second are only used to distinguish one entity or operation from another entity or operation, and do not necessarily require or imply any such actual relationship or sequence between these entities or operations. Moreover, the terms “comprise”, “include” or any other variants intended to cover non-exclusive inclusion, so that a process, method, commodity or equipment comprising elements not only comprise those elements, but also comprise other unlisted elements, or also comprise elements inherent to this process, method, commodity or equipment. In a situation with no more restrictions, the element defined by the sentence “comprising a . . . ” does not exclude the existence of other identical elements in the process, method, commodity or equipment that comprises the element.

The above is a detailed introduction to a method for drug recommendation, a device for drug recommendation, and an electronic device provided by the present disclosure. Specific examples are used in this article to illustrate the principles and implementations of the present disclosure. The description is only used to help understand the methods and core ideas of the present disclosure; at the same time, for those of ordinary skills in the art, according to the ideas of the present disclosure, there may be changes in the specific implementation and scope of application. In summary, the content of this description should not be construed as a limitation of this disclosure.

1. A method for drug recommendation, comprising:
receiving information of a specified disease and a name of a first drug for the specified disease input by a user;
obtaining information of the first drug associated with the name of the first drug and information of a plurality of second drugs associated with the information of the specified disease from a pre-established drug database;
determining a text semantic similarity between the information of the first drug and information of each of the plurality of second drugs;
determining information of a recommended drug from the information of the plurality of second drugs based on the text semantic similarity; and
outputting the information of the recommended drug.

2. The method of claim 1, before receiving the information of the specified disease and the name of the first drug for the specified disease input by the user, further comprising:
obtaining information of a plurality of diseases and drug data for each of the plurality of diseases from a drug information source; and
for each of the plurality of diseases, extracting information of a drug for the disease from the drug data, and storing information of the disease in association with the information of the drug for the disease to establish the drug database.

3. The method of claim 2, wherein the information of the drug comprises at least one of a name of the drug, efficacy information of the drug and chemical composition information of the drug.

4. The method of claim 3, wherein the information of the drug comprises the name of the drug, the efficacy information of the drug and the chemical composition information of the drug, and storing the information of the disease in association with the information of the drug for the disease comprises:
setting a disease identification of the disease for the information of the disease and setting a drug identification of each drug for the disease for a name of said each drug for the disease;
establishing a mapping table between the disease identification and the drug identification for each drug; and
storing the drug identification for each drug in the mapping table and the efficacy information and the chemical composition information of the drug in form of a dictionary.

5. The method of claim 4, wherein obtaining the information of the first drug associated with the name of the first drug and the information of the plurality of second drugs associated with the information of the specified disease from the pre-established drug database comprises:
in the pre-established drug database, searching for a drug identification for the first drug based on the name of the first drug, and obtaining efficacy information and chemical composition information of the first drug based on the drug identification for the first drug; and
searching for a disease identification for the specified disease based on the information of the specified disease, determining drug identifications for a plurality of second drugs associated with the disease identification for the specified disease, and obtaining a name, efficacy information and chemical composition information of each of the plurality of second drugs based on drug identification for said each of the plurality of second drugs.

6. The method of claim 5, wherein determining the text semantic similarity between the information of the first drug and the information of each of the plurality of second drugs comprises:
calculating an efficacy similarity between the efficacy information of the first drug and the efficacy information of said each second drug;
calculating a composition similarity between the chemical composition information of the first drug and the chemical composition information of said each second drug; and
calculating the text semantic similarity between the information of the first drug and the information of said each second drug according to the efficacy similarity and the composition similarity.

7. The method of claim 6, wherein the efficacy information of the first drug comprises text describing efficacy of the first drug, the efficacy information of the second drug comprises text describing efficacy of the second drug, and calculating the efficacy similarity between the efficacy information of the first drug and the efficacy information of said each second drug comprises:
performing word segmentation and keyword recognition processing on the text describing the efficacy of the first drug to generate a first word vector sequence of a first keyword for the efficacy information of the first drug;
performing word segmentation and keyword recognition processing on the text describing efficacy of said each second drug to generate a second word vector sequence of a second keyword for the efficacy information of said each second drug; and
inputting the first word vector sequence and the second word vector sequence into a first neural network model, to calculate the efficacy similarity.

8. The method of claim 6, wherein the chemical composition information of the first drug comprises text describing chemical composition of the first drug, the chemical composition information of the second drug comprises text describing chemical composition of the second drug, and calculating the composition similarity between the chemical composition information of the first drug and the chemical composition information of said each second drug comprises:
performing word segmentation and keyword recognition processing on the text describing the chemical composition of the first drug to generate a third word vector sequence of a third keyword for the chemical composition information of the first drug;
performing word segmentation and keyword recognition processing on the text describing the chemical composition of said each second drug to generate a fourth word vector sequence of a fourth keyword for the chemical composition information of said each second drug; and
inputting the third word vector sequence and the fourth word vector sequence into a second neural network model, to calculate the composition similarity.

9. The method of claim 6, wherein calculating the text semantic similarity between the information of the first drug and the information of said each second drug according to the efficacy similarity and the composition similarity comprises:
calculating an average value of the efficacy similarity and the composition similarity between the first drug and said each second drug, as the text semantic similarity between the information of the first drug and the information of said each second drug.

10. The method of claim 1, wherein determining the information of the recommended drug from the information of the plurality of second drugs based on the text semantic similarity comprises:
obtaining a highest similarity of the text semantic similarities to the information of the first drug as a target text semantic similarity; and
from the information of the plurality of second drugs, determining information of a second drug having the target text semantic similarity to the information of the first drug as the information of the recommended drug.

11. The method of claim 1, wherein the information of the specified disease comprises a name of the specified disease.

12. An electronic device comprising:
a processor; and
a memory, configured to store executable instructions for the processor;
wherein, the processor is configured to perform the method for drug recommendation of claim 1.

13. A computer-readable storage medium, storing instructions thereon, wherein the instructions, when executed by a processor, cause the processor to perform the method for drug recommendation of claim 1.

14. The method of claim 7, wherein the chemical composition information of the first drug comprises text describing chemical composition of the first drug, the chemical composition information of the second drug comprises text describing chemical composition of the second drug, and calculating the composition similarity between the chemical composition information of the first drug and the chemical composition information of said each second drug comprises:
performing word segmentation and keyword recognition processing on the text describing the chemical composition of the first drug to generate a third word vector sequence of a third keyword for the chemical composition information of the first drug;
performing word segmentation and keyword recognition processing on the text describing the chemical composition of said each second drug to generate a fourth word vector sequence of a fourth keyword for the chemical composition information of said each second drug; and
inputting the third word vector sequence and the fourth word vector sequence into a second neural network model, to calculate the composition similarity.

15. The method of claim 7, wherein calculating the text semantic similarity between the information of the first drug and the information of said each second drug according to the efficacy similarity and the composition similarity comprises:
calculating an average value of the efficacy similarity and the composition similarity between the first drug and said each second drug, as the text semantic similarity between the information of the first drug and the information of said each second drug.

16. The method of claim 14, wherein calculating the text semantic similarity between the information of the first drug and the information of said each second drug according to the efficacy similarity and the composition similarity comprises:
calculating an average value of the efficacy similarity and the composition similarity between the first drug and said each second drug, as the text semantic similarity between the information of the first drug and the information of said each second drug.

17. The method of claim 2, wherein determining the information of the recommended drug from the information of the plurality of second drugs based on the text semantic similarity comprises:
obtaining a highest similarity of the text semantic similarities to the information of the first drug as a target text semantic similarity; and
from the information of the plurality of second drugs, determining information of a second drug having the target text semantic similarity to the information of the first drug as the information of the recommended drug.

18. The method of claim 3, wherein determining the information of the recommended drug from the information of the plurality of second drugs based on the text semantic similarity comprises:
obtaining a highest similarity of the text semantic similarities to the information of the first drug as a target text semantic similarity; and
from the information of the plurality of second drugs, determining information of a second drug having the target text semantic similarity to the information of the first drug as the information of the recommended drug.

19. An electronic device comprising:
a processor; and
a memory, configured to store executable instructions for the processor;
wherein, the processor is configured to perform the method for drug recommendation of claim 2.

20. A computer-readable storage medium, storing instructions thereon, wherein the instructions, when executed by a processor, cause the processor to perform the method for drug recommendation of claim 2.
